(19) United States
(12) Patent Application Publication
(10) Pub. No.: US 2002/0094988 A1
(43) Pub. Date: Jul. 18, 2002

(54) Method of treating erectile dysfunction

(76) Inventors: Robert Hines, Fayetteville, NC (US); Ernest L. Bonner JR., Alameda, CA (US)

Correspondence Address: Brian Beverly, Suite 2360, One Kaiser Plaza, Oakland, CA, 94612, US

(21) Appl. No.: 10/039,993

(22) Filed: Jan. 4, 2002

Related U.S. Application Data
(63) Continuation of application No. 09/311,985, filed on May 14, 1999, now abandoned.

(51) Int. Cl. A61K 31/496; A61K 31/47

(52) U.S. Cl. 514/252.16; 514/307

(57) ABSTRACT

An improved method of treating male erectile dysfunction by enteral administration of a combination of sildenafil citrate and papavarine hydrochloride. An alternate mode of treatment indicated for individuals with known vascular disease includes the contemporaneous administration of pentoxifylline. Further embodiments include administration of zinc monomethionine aspartate.

This application is a continuation of, and claims the benefit of, U.S. application Ser. No. 09/311,985, filed Jun. 14, 1999, and of the Continued Prosecution Application filed 2/21/01.

### FIELD OF THE INVENTION

This invention relates to methods for treatment of erectile dysfunction and more particularly to treating organically caused male erectile dysfunction with a combination of drugs.

### DESCRIPTION OF THE RELATED ART

Erectile dysfunction can be defined as a male's inability to attain a sufficiently strong erection to enable him satisfactorily to engage in sexual intercourse. Conventionally it was believed that the majority of men with erectile dysfunction had a psychogenic etiology for their impotence. It is currently felt that a majority of impotent men have a component of underlying organic disease. Typically, erectile dysfunction results from a combination of psychogenic and organic factors. It is estimated that ten to twelve million men in the United States between the ages of 18 and 75 suffer from chronic impotence, the great majority being over the age of 55.

The organic causes of erectile dysfunction can be grouped into five areas: endocrine causes, drugs, penile diseases, neurologic diseases, and vascular diseases. Medical therapy for erectile dysfunction with androgens offers little more benefit than placebos except in hypogonadal men. If a prolactin-secreting pituitary tumor is found, it may be surgically removed or treated with bromocriptine which usually results in return of potency. Surgical therapy may also be useful in treatment of decreased potency related to aortic obstruction, but involves the risk that potency can be lost rather than improved if the autonomic nerve supply to the penis is damaged. The efficacy of penile revascularization and balloon embolization for vasculogenic impotency remains uncertain. Men with primary venous leak impotency, without associated arterial or sinusoidal disease, may benefit from venous ligation.

A variety of vasoactive substances produce erection when injected into the corpora cavernosa. For example, Latorre, U.S. Pat. No. 4,127,118, discloses a method of alleviating and treating male impotence by effecting and enhancing an erection by injecting into the penis an appropriate vasodilatory, a sympathomimetic amine, or an adrenergic blocking agent. Self-injection with papaverine hydrochloride, with or without phentolamine, produces erection in patents with psychogenic, neurogenic, and mild vasculogenic impotency. Lack of FDA approval, pain on injection, and possible complications of priapism and penile fibrosis limit the use of papaverine hydrochloride.

Pharmacological advances have provided relatively recent treatment alternatives. Laragh, U.S. Pat. No. 5,399,581, discloses a method and compositions for treating sexual impotence with an oral drug regimen combining the administration of a non-selective alpha₁-alpha₂ adrenergic blocking drug, such as dibenzyline, with that of a particular type of beta adrenergic blocking agent which also possesses vasodilator activity, such as labetalol, celiprolol, or carvedilol. The side-effects of such a combination limits its usefulness to a large number of individuals, including those with chronic obstructive lung disease, diabetes, and heart disease. Omar, U.S. Pat. No. 5,730,987, discloses a composition for treating impotence in males including a mixture of lyophilized roe and a dry powdered extract from leaves of Ginkgo biloba. Milne, U.S. Pat. No. 5,270,323, discloses a method of relieving erectile impotence in a human male comprising administering a compound selected from the group consisting of U.K. 52,046, Amlodipine, Doxazosin and the pharmaceutically acceptable acid addition salts thereof Commercially available mechanical devices that utilize a vacuum to produce an erection and a rubber band or ring to restrict venous return at the base of the penis, provide a successful nonsurgical alternative in many patients, including some with diabetes mellitus.

Penile prostheses are the most common therapeutic alternative in impotent patients refractory to other forms of therapy. Malleable silastic rods implanted into the penis provide the simplest system and the lowest complication rates. However, the cosmetic and functional performance of these devices is not uniformly satisfactory. Multicomponent, hydraulically operated prostheses offer the advantage of more physiologic erection and greater increase in penile diameter, but these devices are subject to mechanical failure.

Psychotherapy is often beneficial in alleviating psychogenic factors that limit the success of medical and surgical therapy, even in patients with organic impotence.

### SUMMARY OF THE INVENTION

The invention is directed to an improved method of treating male erectile dysfunction by enteral administration of a combination of sildenafil citrate and papavarine hydrochloride. An alternate mode of treatment includes the contemporaneous administration of pentoxifylline where indicated in patients with known vascular disease. Other modes of treatment include, either with or without pentoxifylline, the contemporaneous enteral administration of zinc monomethionine aspartate.

### DETAILED DESCRIPTION OF THE INVENTION

The invention is an improved method of treating male erectile dysfunction by administering a combination of sildenafil citrate, popularly known under the brand name Viagra®, and papavarine hydrochloride, available under the brand name Pavabid®. A second mode of treatment appropriate for individuals with known vascular disease includes the administration of pentoxifylline, commercially available under the brand name Trental®. Further embodiments include the administration of zinc.

It is now understood that sildenafil citrate is an effective and safe inhibitor of phosphodiesterase, the predominant isoenzyme in the human corpus cavernosum. Decreased levels of phosphodiesterase allow increased production of nitrous oxide which, in turn, stimulates blood flow to the corpus cavernosum. An effective dose of sildenafil citrate is usually between 25 and 100 milligrams. Used alone, sildenafil citrate has been found to improve erectile function in a large percentage of males with erectile dysfunction. Of those individuals experiencing improved erections using sildenafil citrate, the improvement in some has been significant, while the improvement in others has been modest to minor. A significant minority of individuals experience no improvement with use of sildenafil citrate.

Papaverine hydrochloride relaxes the smooth musculature of the large blood vessels, especially coronary, pulmonary and systemic peripheral arteries. Conventional medical wisdom is that papaverine hydrochloride is not indicated for treatment of erectile dysfunction because the usual method in the art has been to deliver papaverine hydrochloride into the penis by intracorporeal injection. The intracorporeal injection of papaverine hydrochloride has been reported to have resulted in persistent priapism requiring medical and surgical intervention. However, in preliminary trials applicants have observed surprising beneficial results in individuals treated with a combination of sildenafil citrate and enterally administered papaverine hydrochloride with no observed incidents of priapism or other significant adverse side effects. A normal dosage of papaverine hydrochloride for oral administration is between fifty and four hundred fifty milligrams daily, with the usual dosage being one hundred fifty milligrams taken twice daily. Any non-toxic amount of sildenafil citrate and of papaverine hydrochloride that is effective in relieving male erectile dysfunction may be used.

Pentoxifylline is indicated for treatment of individuals with intermittent claudication stemming from chronic occlusive arterial disease of the limbs. It improves the flow properties of blood by decreasing its viscosity. In patients with chronic peripheral arterial disease, this increases blood flow to the affected microcirculation and enhances tissue oxygenation. It is not known to use pentoxifylline as a treatment for erectile dysfunction. Nevertheless, unexpected and dramatic improvements in the ability to achieve and sustain a natural erection have been reported by males with erectile dysfunction who have been treated with a combination of sildenafil citrate, papaverine hydrochloride and pentoxifylline. Accordingly, in individuals having peripheral vascular disease, the preferred mode of treatment for erectile dysfunction is a combination of sildenafil citrate, papaverine hydrochloride and pentoxifylline. A normal dosage of pentoxifylline is 400 milligrams taken twice daily. However, an effective dose when taken in combination with sildenafil citrate and papaverine hydrochloride may range from 200 milligrams to 1200 milligrams. It will typically require one to seven days before the pentoxifylline becomes effective.

The natural aging process results in decreases in bloodflow to erectile tissue and diminished levels of testosterone. Individuals suffering from erectile dysfunction with organic etiology commonly have decreased bloodflow to erectile tissue and diminished testosterone levels. The subject invention provides a surprisingly effective treatment which alleviates the problem of lowered bloodflow. Administration of zinc provides a complementary benefit by increasing testosterone levels.

Zinc has been shown in a number of studies to increase endogenous production of testosterone. Zinc is most effectively delivered into the body by oral administration of zinc monomethionine aspartate (ZMA). ZMA is a combination of zinc, monomethionine (L-form) aspartate, magnesium aspartate, and pyridoxine. The monomethionine aspartate increases absorption of zinc thus increasing its availability for production of endogenous free testosterone. This method of treatment using a combination of ZMA and (a) sildenafil citrate and papaverine hydrochloride or (b) sildenafil citrate, papaverine hydrochloride and pentoxifylline provides an effective remedy for male erectile dysfunction by increasing blood flow to erectile tissue and by increasing testosterone levels. Depending on the individual, it may take one to six weeks to increase testosterone to higher levels as a result of treating with ZMA.

### EXAMPLE 1

A forty-nine year old male, seen with erectile dysfunction, was rated erectile strength of 3 on a unilinear scale of 1 to 10. Patient was placed on Viagra resulting in improvement to 7. When placed on a combination of Viagra, Pavabid and ZMA, erectile strength improved to 10.

### EXAMPLE 2

A forty-eight year old male with erectile dysfunction was seen who prior to treatment rated erectile strength of 2 on a unilinear scale of 1 to 10. When placed on Pavabid and Trental, erectile strength improved to 8. Subsequent discontinuation of Pavabid and Trental and placed on Viagra alone. Erectile strength decreased to 6. Thereafter placed on Pavabid, Trental and Viagra with improvement of erectile strength to 12.

The drugs discussed above are preferably administered enterally, preferably in combination in a pharmaceutically acceptable vehicle, such as a tablet. Other methods of administration will be readily evident to those skilled in the art. Although particular dosages have been discussed herein, effective dosages may be determined by one of ordinary skill in the art based on particular dosages appropriate for other uses combined with empirical observations.

There have thus been described certain preferred methods of treatment for male erectile dysfunction. While preferred embodiments have been described and disclosed, it will be recognized by those with skill in the art that modifications are within the true spirit and scope of the invention. The appended claims are intended to cover all such modifications.

I claim:

1. An improved method for treating male erectile dysfunction comprising:
administering to a male patient an effective amount of the combination of sildenafil citrate and papaverine hydrochloride, said papaverine hydrochloride administered enterally.

2. The treatment method of claim 1 wherein:
sildenafil citrate is administered in dosages of between approximately 25 and approximately 100 milligrams approximately every four to six hours, and
papavarine hydrochloride is administered enterally in dosages of between approximately 50 and approximately 450 milligrams per day.

3. The treatment method of claim 2 including:
the contemporaneous administration of between approximately 200 milligrams and approximately 1,200 milligrams of pentoxifylline per day.

4. The treatment method of claim 2 wherein:
approximately 100 milligrams of sildenafil citrate is administered.

5. The treatment method of claim 2 wherein:
approximately 150 milligrams of papavarine hydrochloride is administered twice a day.

6. The treatment method of claim 4 wherein:
approximately 150 milligrams of papavarine hydrochloride is administered twice a day.

7. The treatment method of claim 1 further comprising:
an effective amount of pentoxifylline.

8. The treatment method of claim 7 wherein
between approximately 200 milligrams and approximately 1,200 milligrams per day of pentoxifylline is administered.

9. The treatment method of claim 8 wherein:
approximately 400 milligrams of pentoxifylline is administered per day.

10. The treatment method of claim 4 including:
the contemporaneous administration of approximately 400 milligrams of pentoxifylline three times a day.

11. The treatment method of claim 5 including:
the contemporaneous administration of approximately 400 milligrams of pentoxifylline three times a day.

12. The treatment method of claim 6 including:
the contemporaneous administration of between approximately 200 milligrams and approximately 1,200 milligrams of pentoxifylline per day.

13. The treatment method of claim 12 wherein:
approximately 400 milligrams of pentoxifylline is administered three times a day.

14. The treatment method of claim 1 further comprising:
an effective amount of zinc monomethionine aspartate.

15. The treatment method of claim 14 wherein:
between approximately 25 and approximately 75 milligrams per day of zinc monomethionine aspartate is administered per day.

16. The treatment method of claim 15 wherein:
approximately 25 milligrams per day of zinc monomethionine aspartate is administered.

17. The treatment method of claim 4 wherein:
approximately 25 milligrams per day of zinc monomethionine aspartate is administered.

18. The treatment method of claim 5 wherein:
approximately 25 milligrams per day of zinc monomethionine aspartate is administered.

19. The treatment method of claim 6 wherein:
approximately 25 milligrams per day of zinc monomethionine aspartate is administered.

20. The treatment method of claim 10 wherein:
approximately 25 milligrams per day of zinc monomethionine aspartate is administered.

21. The treatment method of claim 11 wherein:
approximately 25 milligrams per day of zinc monomethionine aspartate is administered.

22. The treatment method of claim 12 wherein:
approximately 25 milligrams per day of zinc monomethionine aspartate is administered.

23. The treatment method of claim 13 wherein:
approximately 25 milligrams per day of zinc monomethionine aspartate is administered.

24. An improved method for treating male erectile dysfunction comprising:
administering to a male patient an effective amount of the combination of sildenafil citrate, papavarine hydrochloride, pentoxifylline and zinc monomethionine aspartate.

25. An improved method for treating male erectile dysfunction comprising:
administering to a male patient the combination of
sildenafil citrate in dosages of between 25 and approximately 100 milligrams during a period of between approximately four and approximately six hours,
papavarine hydrochloride, administered enterally, in dosages of between approximately 50 and approximately 450 milligrams per day, and
between approximately 200 milligrams and approximately 1,200 milligrams of pentoxifylline per day.

26. The treatment method of claim 25 including:
the contemporaneous administration of between approximately 25 and approximately 75 milligrams per day of zinc monomethionine aspartate.

27. An improved method for treating male erectile dysfunction comprising:
administering to a male patient the combination of
sildenafil citrate in dosages of between 25 milligrams and approximately 100 milligrams approximately every four to six hours,
papavarine hydrochloride, administered enterally, in dosages of between approximately 50 milligrams and approximately 450 milligrams per day,
pentoxifylline in dosages of between approximately 200 milligrams and approximately 1,200 milligrams per day, and
zinc monomethionine aspartate in dosages of between approximately 25 milligrams and approximately 75 milligrams per day.
